(19) United States
(12) Patent Application Publication
(10) Pub. No.: US 2025/0302804 A1
(43) Pub. Date: Oct. 2, 2025

(54) RESOMELAGON AND ITS DERIVATIVES FOR THE TREATMENT OF CARDIOVASCULAR DISEASE, HYPERTENSION AND ATHEROSCLEROSIS

(71) Applicant: SYNACT PHARMA ApS, Holte (DK)

(72) Inventor: Thomas Engelbrecht Nordkild JONASSEN, Holte (DK)

(21) Appl. No.: 18/864,251

(22) PCT Filed: May 12, 2023
(86) PCT No.: PCT/EP2023/062795

§ 371 Date: Nov. 8, 2024

(30) Foreign Application Priority Data
May 12, 2022 (EP) 22172903.1

(51) Int. Cl. A61K 31/402; A61P 9/12; A61P 19/02; A61P 29/00

(52) U.S. Cl. CPC A61K 31/402; A61P 9/12; A61P 19/02; A61P 29/00

(57) ABSTRACT

Provided is a composition comprising a phenyl pyrrole aminoguanidine derivative for use in a method of treating cardiovascular disease, hypertension and/or atherosclerosis.

### TECHNICAL FIELD

The present invention relates to a composition comprising a compound of formula (I), or (II), or a pharmaceutically acceptable derivative thereof, for use in a method of treating cardiovascular disease, hypertension and/or atherosclerosis.

### BACKGROUND

The melanocortin system is a set of neuropeptidergic and immunoendocrine signaling pathways that play an integral role in the homeostatic control of a diverse array of physiological functions, including melanogenesis, stress response, inflammation, immunomodulation and adrenocortical steroidogenesis. It consists of multiple components, including the five G protein-coupled melanocortin receptors: melanocortin receptor 1 (MC1R) to MC5R; peptide ligands: α, β, γ-melanocyte stimulating hormone (α, β, γ-MSH), adrenocorticotropic hormone (ACTH) secreted by the anterior pituitary; and endogenous antagonists. The biological functions of melanocortin system are mediated by the five melanocortin receptors (MCRs), which have distinct tissue distribution, convey different signalling and exert varying biological activities in different organ systems. Activation of the MC3R and MC4R has been demonstrated to increase blood pressure (Nordheim et al 2006 peptides 27 (2006) 438-443; White et al., Journal of Hypertension 2017, 35:761-768).

High blood pressure (hypertension) is a common condition that affects the body's arteries; the force of the blood pushing against the artery walls is consistently too high and the heart has to work harder to pump blood. In general, hypertension is a blood pressure of 130/80 mm Hg or higher. Untreated, high blood pressure causes cardiac hypertrophy and increases the risk of heart attack (myocardial infarction), stroke and other serious health problems. Hypertension is the most widespread modifiable risk factor for development of atherosclerosis.

Atherosclerosis, the accumulation of cholesterol and immune cells in the vascular wall, is the most common underlying cause of numerous cardiovascular diseases, which often lead to morbidity and mortality. Recent research shows that melanocortin peptides have a protective effect and can prevent atherosclerosis (Rinne et al. Arterioscler Thromb Vasc Biol. 2018; 38:).

Rheumatoid arthritis (RA) is an autoimmune disorder that primarily affects joints and some 0.5-1% of adults in the developed world are affected by RA. While the cause of rheumatoid arthritis is not clear, it is believed to involve a combination of genetic and environmental factors. The goal of current treatments is to reduce pain and inflammation to improve the quality of life of the patients suffering from the condition. Pain medications, steroids, and non-steroidal anti-inflammatory drugs (NSAIDs) are frequently used as treatment to reduce symptoms. Disease-modifying antirheumatic drugs (DMARDs), such as hydroxychloroquine and in particular methotrexate (MTX), may be employed in an attempt to slow down the progression of disease.

A large proportion of premature deaths in RA patients are due to cardiovascular comorbidities. For instance, the incidence of heart failure in RA patients is twice as high compared to the general population, and RA patients thus are susceptible to heart failure. None of the known therapies for RA targeting the joints are also known to protect the heart.

Phenyl pyrrole aminoguanidine derivatives with activity on the melanocortin receptors are disclosed in WO 2007/141343. One example of such compound is the anti-inflammatory AP1189 ((E)-N-trans-{3-[1-(2-nitrophenyl)-1H-pyrrol-2-yl]-allylidene}-aminoguanidium acetate) which was first shown to bind the MC1R (WO 2007/141343) and later was identified as a biased dual agonist at receptors MC1R and MC3R that does not provoke canonical cAMP generation (Montero-Melendez et al. 2015). AP1189 is a clinical candidate showing promising efficacy for treatment of rheumatoid arthritis.

### SUMMARY

The present inventors have found that the phenyl pyrrole aminoguanidine derivative resomelagon or ‘AP1189’ ((E)-N-trans-{3-[1-(2-nitrophenyl)-1H-pyrrol-2-yl]-allylidene}-aminoguanidium acetate) reduces the levels of pro-inflammatory cytokines IL-1 and IL-1B, and reduces neutrophil accumulation, as compared to vehicle. Furthermore, the inventors have demonstrated that resomelagon reduces blood pressure and reduces the number of observed abnormalities in blood pressure. This implies that resomelagon and related compounds are candidates for the treatment of hypertension, atherosclerosis and cardiovascular diseases, potentially by helping the immune system to dissolve inflammation in the vessels and/or via an effect on blood pressure. In the treatment of rheumatoid arthritis, where cardiovascular disease is a known and frequent comorbidity, it is of particular importance to avoid exacerbating cardiovascular abnormalities and ensure that treatment-which is often chronic-does not increase blood pressure.

It is an aspect of the present disclosure to provide a composition comprising a compound of formula (I):

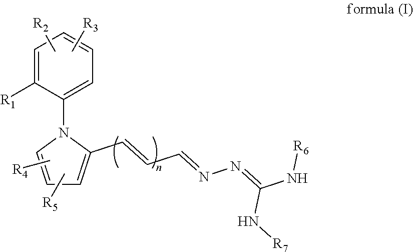

including tautomeric and stereoisomeric forms thereof;
wherein n is 1; and R₁ is CF₃, CCl₃, F, Cl, NO₂ or CN, and R₂, R₃, R₄, R₅, R₆, and R₇ are hydrogen; or a pharmaceutically acceptable derivative thereof,
for use in the treatment of cardiovascular disease, hypertension and/or atherosclerosis.

In one embodiment said compound is selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, such as acetate, succinate, tartarate or propionate.

In one embodiment said compound is (E)-N-trans-{3-[1-(2-nitrophenyl)-1H-pyrrol-2-yl]-allylidene}-aminoguanidinium acetate (resomelagon, or AP1189) or (E)-N-trans-{3-[1-(2-nitrophenyl)-1H-pyrrol-2-yl]-allylidene}-aminoguanidinium succinate.

### DESCRIPTION OF DRAWINGS

Test Item A=AP1189 (E)-N-trans-{3-[1-(2-nitrophenyl)-1H-pyrrol-2-yl]-allylidene}-aminoguanidium acetate.

FIG. 1: Pre-treatment with AP1189 reduces the levels of the pro-inflammatory cytokines IL-1ß compared to pretreatment with vehicle by 37%.

FIG. 2: Pre-treatment with AP1189 reduces the levels of the pro-inflammatory cytokines IL-6 compared to pretreatment with vehicle by 59%.

FIG. 3: Pre-treatment with AP1189 reduces neutrophil accumulation compared to pretreatment with vehicle by 70%.

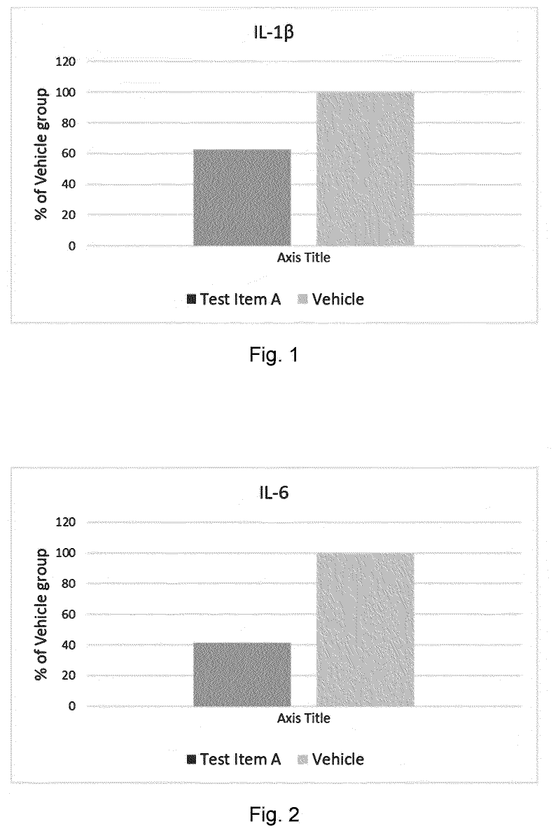

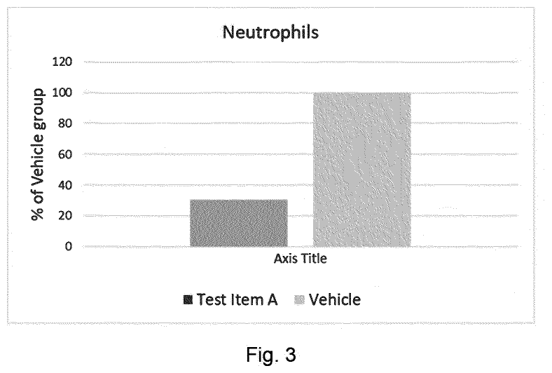

### Definitions

The term “pharmaceutically acceptable derivative” in the present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes esters and prodrugs, or other precursors of a compound which may be biologically metabolized into the active compound, or crystal forms of a compound.

The term “acid addition salt” is intended to include “pharmaceutically acceptable acid addition salt” which indicates salts which are not harmful to the patient. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The terms “treatment” and “treating” as used herein refer to the management and care of a patient for the purpose of combating a condition, disease or disorder. The term is intended to include the full spectrum of treatments for a given condition from which the patient is suffering. The individual to be treated is preferably a mammal, in particular a human being. Treatment of animals, such as mice, rats, dogs, cats, horses, cows, sheep and pigs, is, however, also within the scope of the present context. The individual to be treated can be of various ages.

In some embodiments, the term “about” refers to the recited amount, value, or duration ±10%, ±9%, ±8%, ±7%, ±6%, ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5%±0.4%, ±0.3%, ±0.2% or ±0.1%.

### DETAILED DESCRIPTION

Melanocortin (MC) receptors (MC1R-MC5R), a family of class A G protein-coupled receptors (GPCRs), are attractive therapeutic targets for a number of conditions due to their wide distribution and diversity of physiological processes they regulate. MC1R regulates UV light-induced skin tanning and other immune responses because of its expression on leukocytes. MC2R regulates cortisol production on the adrenal glands, whereas MC5R plays a role on exocrine glands secretions. MC3R and MC4R exert non-redundant functions on energy homeostasis in addition to specific anti-inflammatory roles; whereas MC3R activation is particularly protective for joint inflammation such as arthritis, MC4R provides neuroprotection in brain inflammation. Accordingly, an array of pathological situations could be targeted with MCR-drugs including skin conditions, cardiovascular pathologies, joint inflammation, obesity and cachexia.

Peripheral MC1R and MC3R can be pharmacologically activated to induce anti-inflammation. The endogenous agonist α-melanocyte-stimulating hormone (αMSH), like other protective mediators, is released by immune cells to counterbalance proinflammatory signals, thus preventing excessive tissue damage. In line with the resolution of inflammation concept, therapeutics targeting MC1R and MC3R act by mimicking the body's own protective resources and might be characterized by a lighter burden of side effects. Activation of the MC3R and MC4R has been demonstrated to increase blood pressure (Nordheim et al 2006 peptides 27 (2006) 438-443; White et al., Journal of Hypertension 2017, 35:761-768).

Shown to be effective in rheumatic diseases since the early 1950s, the use of corticotropin or adrenocorticotropin hormone (ACTH) declined when synthetic glucocorticoids became available. However, the discovery of an alternative anti-inflammatory mechanism for ACTH involving activation of peripheral MC receptors on immune cells has revived the interest in developing novel ACTH-like molecules with no steroidogenic effects for the treatment of joint diseases such as gout or RA (rheumatoid arthritis). However, the limitation in the translational delivery of novel MC drugs besides the marketed ACTH formulations is imposed by the lack of receptor selectivity achieved so far.

Innovative approaches in G protein-coupled receptor drug discovery might help to overcome this limitation. Allosteric modulation consists in the ability of a molecule to enhance (positive modulation) or reduce (negative modulation) the effect of the endogenous ligand by binding to a distinct site of the receptor protein, termed allosteric site. A higher degree of selectivity is expected as allosteric regions are less conserved among the five MCRs, and indeed, allosteric modulators at MC4R are currently under development for the treatment of obesity.

Another emerging concept of significant therapeutic interest is the one of biased agonism. The obsolete notion that receptors could exist in two unique conformations, the active one and the inactive one, has been replaced with the conception that multiple active conformations can exist, each one creating a distinct signal yielding multiple functional outcomes. Receptor activation, rather than linear and static, is emerging as a highly dynamic and multidimensional process in which a diversity of active conformations may be induced by different molecules leading to distinct effects.

The small molecule resomelagon or AP1189 ((E)-N-trans-{3-[1-(2-nitrophenyl)-1H-pyrrol-2-yl]-allylidene}-aminoguanidium acetate) has been characterized as a biased agonist at receptors MC1R and MC3R, which does not induce canonical CAMP generation, but cause ERK1/2 phosphorylation, a signaling responsible for the proefferocytic effect evoked in mouse primary macrophages. AP1189 was shown to reduce cytokine release in macrophages, whereas no melanogenesis was induced by AP1189 in melanocytes. In vivo, oral AP1189 elicits anti-inflammatory actions in peritonitis and accelerated the resolution phase, and afforded significant reduction of macroscopic and histological parameters of joint disruption in experimental inflammatory arthritis. AP1189 is thus a biased dual agonist at MC1R and MC3R with anti-inflammatory properties together with a lack of effect on melanogenesis.

Rheumatoid arthritis (RA) is an autoimmune disorder that primarily affects joints. RA patients suffer a higher risk of premature deaths due to cardiovascular comorbidities than the general population. None of the known therapies for RA targeting the joints are known to also protect the heart.

In RA patients it is of particular importance not to increase the risk of cardiovascular comorbidities when administrating a long-term therapy. The compound of the present invention is shown herein to stabilise cardiovascular abnormalities and reduce blood pressure in normotensive patients. These compounds thus hold the promise to be effective in RA without increasing cardiovascular comorbidities, and potentially reducing the risk of developing cardiovascular comorbidities.

Hypertension, also known as high blood pressure (HBP), is a long-term medical condition in which the blood pressure in the arteries is persistently elevated. High blood pressure usually does not cause symptoms, it is however a major risk factor for stroke, coronary artery disease, heart failure, atrial fibrillation, peripheral arterial disease, vision loss, chronic kidney disease, and dementia. Hypertension is a major cause of premature death worldwide.

High blood pressure is classified as primary (essential) hypertension or secondary hypertension. About 90-95% of cases are primary, defined as high blood pressure due to nonspecific lifestyle and genetic factors. Lifestyle factors that increase the risk include excess salt in the diet, excess body weight, smoking, physical inactivity and alcohol use. The remaining 5-10% of cases are categorized as secondary high blood pressure, defined as high blood pressure due to an identifiable cause, such as chronic kidney disease, narrowing of the kidney arteries, an endocrine disorder, or the use of birth control pills.

Blood pressure is classified by two measurements, the systolic and diastolic pressures, which are the maximum and minimum pressures, respectively. For most adults, normal blood pressure at rest is within the range of 100-130 millimeters mercury (mmHg) systolic and 60-80 mmHg diastolic. For most adults, elevated blood pressure is defined as a systolic blood pressure ranges from 120 to 129 mmHg with a diastolic blood pressure at or below 80 mmHg. High blood pressure—hypertension—is present if the resting blood pressure is persistently at or above 130/80 mmHg (stage 1 hypertension) or 140/90 mmHg (stage 2 hypertension). Different numbers apply to children. Ambulatory blood pressure monitoring over a 24-hour period appears more accurate than office-based blood pressure measurement.

Hypertension is a major risk factor for development of atherosclerosis. Atherosclerosis is a pattern of the disease arteriosclerosis in which the wall of the artery develops abnormalities, called lesions. These lesions may lead to narrowing due to the buildup of atheromatous plaque (fatty deposits).

Atherosclerosis is a chronic disease that may be asymptotic for years. Atherosclerosis increases the risk of thrombus formation in the lumen or complete closure of the lumen (stenosis) of vessels, of thromboembolism, and resulting ischemia or infarction. When severe, it can result in coronary artery disease, stroke, peripheral artery disease, or kidney problems, depending on which arteries are affected. Accompanying incidents include myocardial infarction (a heart attack due to thrombosis of a coronary artery), stroke (thrombosis of cerebral and/or carotid arteries), claudication from insufficient blood supply to the legs, and thrombosis of renal and femoral arteries.

Risk factors include abnormal cholesterol levels, elevated levels of inflammatory markers, high blood pressure, diabetes, smoking, obesity, family history, and an unhealthy diet Plaque is made up of fat, cholesterol, calcium, and other substances found in the blood. The narrowing of arteries limits the flow of oxygen-rich blood to parts of the body. Diagnosis is based upon a physical exam, electrocardiogram, and exercise stress test, among others.

Treatment of established disease may include medications to lower cholesterol such as statins, blood pressure medication, or medications that decrease clotting, such as aspirin. A number of procedures may also be carried out such as percutaneous coronary intervention, coronary artery bypass graft, or carotid endarterectomy.

Atherosclerosis is associated with inflammatory processes in the endothelial cells of the vessel wall associated with retained low-density lipoprotein (LDL) particles. This retention may be a cause, an effect, or both, of the underlying inflammatory process.

Once inside the vessel wall, LDL particles can become more prone to oxidation. Endothelial cells respond by attracting monocyte white blood cells, causing them to leave the blood stream, penetrate into the arterial walls and transform into macrophages. The accumulation of the white blood cells is termed “fatty streaks” early on. The macrophages' ingestion of oxidized LDL particles triggers a cascade of immune responses which over time can produce an atheroma if HDL removal of fats from the macrophages does not keep up. The immune system's specialized white blood cells (macrophages and T-lymphocytes) absorb the oxidized LDL, forming specialized foam cells. If these foam cells are not able to process the oxidized LDL and recruit HDL particles to remove the fats, they grow and eventually rupture, leaving behind cellular membrane remnants, oxidized materials, and fats (including cholesterol) in the artery wall. This attracts more white blood cells, resulting in a snowballing progression that continues the cycle, inflaming the artery.

Cardiovascular disease (CVD) is a class of diseases that involve the heart or blood vessels. CVD includes coronary artery diseases (CAD) such as angina and myocardial infarction (commonly known as a heart attack). Other CVDs include stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis.

The underlying mechanisms vary depending on the disease. Coronary artery disease, stroke (cerebrovascular disease) and peripheral artery disease involve atherosclerosis, and hence these can be classified as atherosclerotic cardiovascular disease (ASCVD).

It is an aspect of the present disclosure to provide a composition comprising a compound of formula (I):

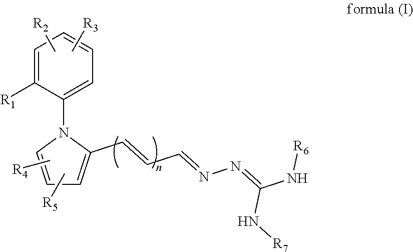

including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen;
or a pharmaceutically acceptable derivative thereof,
for use in the treatment of cardiovascular disease, hypertension and/or atherosclerosis.

The term as used herein throughout “for use in the treatment of cardiovascular disease, hypertension and/or atherosclerosis” includes both the treatment of the disease or condition, preventing the disease or condition, reducing the risk of developing the disease or condition, as well as ameliorating the disease or condition.

It is an aspect of the present disclosure to provide a compound of formula (I), including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen; or a pharmaceutically acceptable derivative thereof, for use in the treatment of cardiovascular disease, hypertension and/or atherosclerosis.

It is also an aspect to provide the use of a composition comprising a compound of formula (I):

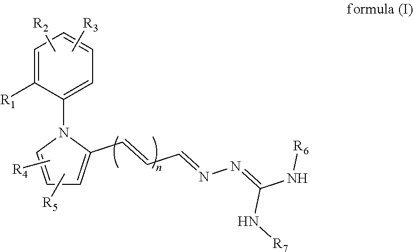

including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen;
or a pharmaceutically acceptable derivative thereof
for the manufacture of a medicament for the treatment of cardiovascular disease, hypertension and/or atherosclerosis.

Also disclosed is a method for treating cardiovascular disease, hypertension and/or atherosclerosis, said method comprising one or more steps of administration of a composition comprising a compound of formula (I):

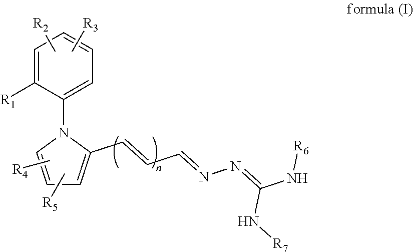

including tautomeric and stereoisomeric forms thereof; wherein n is 1; and
R₁ is CF₃, CCl₃, F, Cl, NO₂ or CN, and R₂, R₃, R₄, R₅, R₆, and R₇ are hydrogen;
or a pharmaceutically acceptable derivative thereof,
to an individual in need thereof.

An individual in need of treatment with the composition according to the present disclosure is on one embodiment an individual with high blood pressure.

An individual in need of treatment with the composition according to the present disclosure is on one embodiment an individual with hypertension, such as an individual with a resting blood pressure at or above 130/80 mmHg (stage 1 hypertension).

An individual in need of treatment with the composition according to the present disclosure is on one embodiment an individual with hypertension, such as an individual with a resting blood pressure at or above 140/90 mmHg (stage 2 hypertension).

An individual in need of treatment with the composition according to the present disclosure is on one embodiment an individual with atherosclerosis.

An individual in need of treatment with the composition according to the present disclosure is on one embodiment an individual with atheromatous plaques.

An individual in need of treatment with the composition according to the present disclosure is on one embodiment an individual with a cardiovascular disease.

An individual in need of treatment with the composition according to the present disclosure is on one embodiment an individual with an increased risk of developing a cardiovascular disease.

In one embodiment an individual in need of treatment with the composition according to the present disclosure is an individual with an arthritic disease,
such as an arthritic disease selected from the group consisting of inflammatory arthritis, degenerative arthritis, metabolic arthritis, reactive arthritis and infectious arthritis, such as wherein the inflammatory arthritis is selected from the group consisting of Rheumatoid Arthritis, Psoriatic Arthritis, and Ankylosing Spondylitis, such as wherein the degenerative arthritis is osteoarthritis, such as wherein the metabolic arthritis is gouty arthritis.

In one embodiment an individual in need of treatment with the composition according to the present disclosure is an individual with rheumatoid arthritis (RA).

In one embodiment an individual in need of treatment with the composition according to the present disclosure is an individual with rheumatoid arthritis with an inadequate response to MTX therapy (DMARD-inadequate response RA).

In one embodiment an individual in need of treatment with the composition according to the present disclosure is an individual with rheumatoid arthritis (RA), wherein said individual with RA:
Has high blood pressure,
Has hypertension, such as stage 1 hypertension or stage 2 hypertension,
Has atherosclerosis and/or atheromatous plagues, and/or
Has an increased risk of developing a cardiovascular disease.

In certain embodiments the composition for use according to the present disclosure comprises a compound of formula (II):

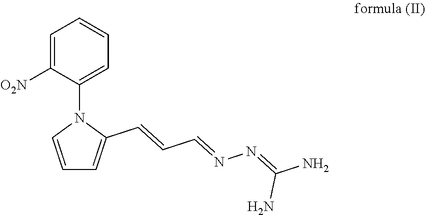 [SMILES: NC(N)=N/N=C/C=C/c1cccn1-c1ccccc1[N+](=O)[O-]]

including tautomeric and stereoisomeric forms thereof; or a pharmaceutically acceptable derivative thereof, for use in the treatment of cardiovascular disease, hypertension and/or atherosclerosis.

It is an aspect of the present disclosure to provide a compound of formula (II), including tautomeric and stereoisomeric forms thereof; or a pharmaceutically acceptable derivative thereof, for use in the treatment of cardiovascular disease, hypertension and/or atherosclerosis.

In a preferred embodiment said composition for use according to the present disclosure comprises (E)-N-trans-{3-[1-(2-nitrophenyl)-1H-pyrrol-2-yl]-allylidene}-aminoguanidinium acetate (AP1189; resomelagon).

In one embodiment said composition for use according to the present disclosure comprises a compound of formula (I) or (II) as defined herein which is:
i) an agonist of one or more MC receptors,
ii) an agonist of the MC1R and MC3R, and/or
iii) a biased agonist of the MC1R and MC3R.

Also disclosed is a composition for use according to the present disclosure comprising a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of cardiovascular disease, hypertension and/or atherosclerosis.

In one embodiment a pharmaceutically acceptable derivative thereof is a pharmaceutically acceptable salt, such as a pharmaceutically acceptable salt of an inorganic acid or an organic acid.

In such embodiments an organic acid as referred to herein is selected from the group consisting of: formic acid, acetic acid, trichloroacetic acid, trifluoroacetic acid, propionic acid, benzoic acid, cinnamic acid, citric acid, fumaric acid, glycolic acid, lactic acid, maleic acid, malic acid, malonic acid, mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid and p-toluenesulfonic acid.

In a particular embodiment said organic acid is acetic acid, succinic acid, tartaric acid or propionic acid; In a particular embodiment said organic acid is acetic acid.

In such embodiments an organic acid as referred to herein is selected from the group consisting of: hydrochloric acid, hydrobromic acid, hydroiodic acid, phosphoric acid, sulphuric acid and nitric acid.

Also disclosed is a composition comprising a compound selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate, for use in the treatment of cardiovascular disease, hypertension and/or atherosclerosis.

In a particular embodiment the present disclosure provides a composition comprising (E)-N-trans-{3-[1-(2-nitrophenyl)-1H-pyrrol-2-yl]-allylidene}-aminoguanidinium acetate for use in the treatment of cardiovascular disease, hypertension and/or atherosclerosis.

Also disclosed is a composition comprising a compound selected from the group consisting of {3-[1-(2-nitrophenyl)-1H-pyrrol-2-yl]-allylidene}-aminoguanidinium succinate and (E)-N-trans-{3-[1-(2-nitrophenyl)-1H-pyrrol-2-yl]-allylidene}-aminoguanidinium succinate, for use in the treatment of cardiovascular disease, hypertension and/or atherosclerosis.

In a particular embodiment the present disclosure provides a composition comprising (E)-N-trans-{3-[1-(2-nitrophenyl)-1H-pyrrol-2-yl]-allylidene}-aminoguanidinium succinate for use in the treatment of cardiovascular disease, hypertension and/or atherosclerosis.

### Hypertension

It is an aspect to provide a composition comprising a compound of formula (I) or formula (II), including tautomeric and stereoisomeric forms thereof, or a pharmaceutically acceptable derivative thereof, according to the present disclosure, for use in the treatment of hypertension.

It is an aspect to provide a composition comprising a compound of formula (I) or formula (II), including tautomeric and stereoisomeric forms thereof, or a pharmaceutically acceptable derivative thereof, according to the present disclosure, for use in the treatment of hypertension in an individual with rheumatoid arthritis and/or cardiovascular comorbidities. Treatment implies treatment, prevention, reducing risk of and amelioration.

In one embodiment said treatment of hypertension comprises reducing blood pressure, such as reducing blood pressure in an individual.

In one embodiment there is provided a composition as defined herein for use in a method of reducing blood pressure.

In one embodiment there is provided a composition as defined herein for use in a method of reducing mean arterial blood pressure (MAP).

In one embodiment there is provided a composition as defined herein for use in a method of reducing systolic arterial blood pressure (SAP).

In one embodiment there is provided a composition as defined herein for use in a method of reducing diastolic arterial blood pressure (DAP).

In one embodiment there is provided a composition as defined herein for use in a method of stabilising blood pressure.

In one embodiment said composition as defined herein reduces blood pressure (BP).

In one embodiment said composition as defined herein reduces blood pressure (BP) in an individual in need thereof. In one embodiment said individual in need thereof is an individual with high blood pressure, an individual with hypertension, an individual with atherosclerosis or atheromatous plaques, such as an individual with a cardiovascular disease or an increased risk of developing a cardiovascular disease, such as in an individual with an arthritic disease.

In one embodiment said composition as defined herein reduces blood pressure (BP) in an individual with rheumatoid arthritis and/or cardiovascular comorbidities.

In one embodiment said composition as defined herein reduces mean arterial blood pressure (MAP).

In one embodiment said composition as defined herein reduces mean arterial blood pressure (MAP) at least about 0.5 mmHg, such as at least about 0.6 mmHg, such as at least about 0.7 mmHg, such as at least about 0.8 mmHg, such as at least about 0.9 mmHg, such as at least about 1.0 mmHg, such as at least about 1.2 mmHg, such as at least about 1.4 mmHg, such as at least about 1.6 mmHg, such as at least about 1.8 mmHg, such as at least about 2.0 mmHg.

In one embodiment said composition as defined herein reduces mean arterial blood pressure (MAP) about 0.5 to about 0.8 mmHg, such as about 0.8 to about 1.0 mmHg, such as about 1.0 to about 1.2 mmHg, such as about 1.2 to about 1.4 mmHg, such as about 1.4 to about 1.6 mmHg, such as about 1.6 to about 1.8 mmHg, such as about 1.8 to about 2.0 mmHg.

In one embodiment said composition as defined herein reduces systolic arterial blood pressure (SAP).

In one embodiment said composition as defined herein reduces systolic arterial blood pressure (SAP) at least about 1.5 mmHg, such as at least about 2.0 mmHg, such as at least about 2.1 mmHg, such as at least about 2.2 mmHg, such as at least about 2.3 mmHg, such as at least about 2.4 mmHg, such as at least about 2.5 mmHg, such as at least about 3.0 mmHg, such as at least about 3.1 mmHg, such as at least about 3.2 mmHg, such as at least about 3.3 mmHg, such as at least about 3.4 mmHg, such as at least about 3.5 mmHg.

In one embodiment said composition as defined herein reduces systolic arterial blood pressure (SAP) about 1.5 to about 2.0 mmHg, such as about 2.0 to about 2.1 mmHg, such as about 2.1 to about 2.2 mmHg, such as about 2.2 to about 2.3 mmHg, such as about 2.3 to about 2.4 mmHg, such as about 2.4 to about 2.5 mmHg, such as about 2.5 to about 3.0 mmHg, such as about 3.0 to about 3.1 mmHg, such as about 3.1 to about 3.2 mmHg, such as about 3.2 to about 3.3 mmHg, such as about 3.3 to about 3.4 mmHg, such as about 3.4 to about 3.5 mmHg.

In one embodiment said composition as defined herein reduces diastolic arterial blood pressure (DAP).

In one embodiment said composition as defined herein reduces diastolic arterial blood pressure (DAP) at least about 0.1 mmHg, such as at least about 0.2 mmHg, such as at least about 0.3 mmHg, such as at least about 0.4 mmHg, such as at least about 0.5 mmHg, such as at least about 0.6 mmHg, such as at least about 0.7 mmHg, such as at least about 0.8 mmHg, such as at least about 0.9 mmHg, such as at least about 1.0 mmHg, such as at least about 1.2 mmHg, such as at least about 1.4 mmHg, such as at least about 1.6 mmHg, such as at least about 1.8 mmHg, such as at least about 2.0 mmHg.

In one embodiment said composition as defined herein reduces diastolic arterial blood pressure (DAP) about 0.1 to about 0.2 mmHg, such as about 0.2 to about 0.3 mmHg, such as about 0.3 to about 0.4 mmHg, such as about 0.4 to about 0.5 mmHg, such as about 0.5 to about 0.6 mmHg, such as about 0.6 to about 0.7 mmHg, such as about 0.7 to about 0.8 mmHg, such as about 0.9 to about 1.0 mmHg.

In one embodiment there is provided a composition as defined herein for use in a method of reducing clinically notable vital sign abnormalities (CNA) (systolic arterial pressure ≥160, <90 mmHg; diastolic arterial pressure ≥105, <50 mmHg).

In one embodiment said composition as defined herein reduces clinically notable vital sign abnormalities (CNA) (systolic arterial pressure ≥160, <90 mmHg; diastolic arterial pressure ≥105, <50 mmHg).

In one embodiment said composition as defined herein reduces the number of observed abnormalities in blood pressure, such as reduces the number of observed abnormalities in blood pressure observed in an individual with RA.

In one embodiment there is provided a composition as defined herein for use in a method of preventing or reducing risk of cardiovascular diseases.

In one embodiment there is provided a composition as defined herein for use in a method of preventing or reducing risk of cardiac hypertrophy.

In one embodiment there is provided a composition as defined herein for use in a method of preventing or reducing risk of coronary artery disease.

In one embodiment there is provided a composition as defined herein for use in a method of preventing or reducing risk of congestive heart failure.

In one embodiment there is provided a composition as defined herein for use in a method of preventing or reducing risk of atrial fibrillation.

In one embodiment there is provided a composition as defined herein for use in a method of preventing or reducing risk of aortic aneurism.

In one embodiment there is provided a composition as defined herein for use in the treatment of hypertensive heart diseases. These include cardiac hypertrophy, coronary artery disease, congestive heart failure, atrial fibrillation and aortic aneurism, as well as atherosclerotic cardiovascular diseases (including coronary artery disease, cerebrovascular disease and peripheral artery disease).

### Cardiovascular Disease & Atherosclerosis

It is an aspect to provide a composition comprising a compound of formula (I) or formula (II), including tautomeric and stereoisomeric forms thereof, or a pharmaceutically acceptable derivative thereof, according to the present disclosure, for use in the treatment of cardiovascular disease.

It is an aspect to provide a composition comprising a compound of formula (I) or formula (II), including tautomeric and stereoisomeric forms thereof, or a pharmaceutically acceptable derivative thereof, according to the present disclosure, for use in the treatment of cardiovascular disease in an individual with rheumatoid arthritis and/or cardiovascular comorbidities. Treatment implies treatment, prevention, reducing risk of and amelioration.

In one embodiment there is provided a composition as defined herein for use in the treatment of cardiovascular disease.

In one embodiment there is provided a composition as defined herein for use in the treatment of a cardiovascular disease selected from the group consisting of coronary artery diseases (CAD) such as angina and myocardial infarction (commonly known as a heart attack); stroke, heart failure, hypertensive heart disease, atherosclerotic heart disease, rheumatic heart disease, cardiac hypertrophy, cardiomyopathy, abnormal heart rhythms, atrial fibrillation, congenital heart disease, congestive heart failure, valvular heart disease, carditis, aortic aneurysms, peripheral artery disease, vascular disease, thromboembolic disease, and venous thrombosis.

In one embodiment said cardiovascular disease is atherosclerotic cardiovascular disease.

It is an aspect to provide a composition comprising a compound of formula (I) or formula (II), including tautomeric and stereoisomeric forms thereof, or a pharmaceutically acceptable derivative thereof, according to the present disclosure, for use in the treatment of atherosclerosis.

It is an aspect to provide a composition comprising a compound of formula (I) or formula (II), including tautomeric and stereoisomeric forms thereof, or a pharmaceutically acceptable derivative thereof, according to the present disclosure, for use in the treatment of atherosclerosis in an individual with rheumatoid arthritis and/or cardiovascular comorbidities. Treatment implies treatment, prevention, reducing risk of and amelioration.

In one embodiment there is provided a composition as defined herein for use in the treatment of atherosclerosis. In one embodiment there is provided a composition as defined herein for use in the prevention of atherosclerosis.

In one embodiment the composition as defined herein is for use in the treatment of atherosclerosis by reducing or preventing atheromatous plaques and/or reducing fatty streaks.

In one embodiment there is provided a composition as defined herein for use in the treatment of atherosclerosis by reducing or preventing atheromatous plaques and/or reducing or preventing fatty streaks.

In one embodiment there is provided a composition as defined herein for use in a method of reducing or preventing atheromatous plaques and/or reducing fatty streaks.

In one embodiment there is provided a composition as defined herein for use in a method of reducing the risk of one or more of thrombus formation, thromboembolism, and resulting ischemia and/or infarction in individuals with atherosclerosis.

In one embodiment there is provided a composition as defined herein for use in a method of reducing the risk of thrombus formation in an individual with atherosclerosis.

In one embodiment there is provided a composition as defined herein for use in a method of reducing the risk of thromboembolism in an individual with atherosclerosis.

In one embodiment there is provided a composition as defined herein for use in a method of reducing the risk of atherosclerotic thrombus formation. In one embodiment there is provided a composition as defined herein for use in a method of preventing atherosclerotic thrombus formation.

In one embodiment there is provided a composition as defined herein for use in a method of reducing the risk of atherosclerotic thromboembolism. In one embodiment there is provided a composition as defined herein for use in a method of preventing atherosclerotic thromboembolism.

In one embodiment there is provided a composition as defined herein for use in a method of reducing the risk of ischemia and/or infarction associated with thrombus formation and thromboembolism in an individual with atherosclerosis.

It is an aspect to provide a composition comprising a compound of formula (I) or formula (II), including tautomeric and stereoisomeric forms thereof, or a pharmaceutically acceptable derivative thereof, according to the present disclosure, for use in the treatment of atherosclerotic cardiovascular disease (ASCVD).

In one embodiment there is provided a composition as defined herein for use in the treatment of atherosclerotic cardiovascular disease (ASCVD).

In one embodiment said atherosclerotic cardiovascular disease is selected from the group consisting of coronary artery disease, stroke (cerebrovascular disease), and peripheral artery disease.

In one embodiment there is provided a composition as defined herein for use in the treatment of coronary artery disease.

In one embodiment there is provided a composition as defined herein for use in the treatment of stroke (cerebrovascular disease).

In one embodiment there is provided a composition as defined herein for use in the treatment of peripheral artery disease.

In one embodiment there is provided a composition as defined herein for use in the treatment of vascular inflammation.

### Combination Therapies

In one embodiment the composition comprising a compound of formula (I) or (II), or a pharmaceutically acceptable derivative thereof as disclosed herein comprises, separately or together, one or more additional active pharmaceutical ingredients.

In one embodiment said one or more additional active pharmaceutical ingredients are used for the treatment of cardiovascular disease, hypertension and/or atherosclerosis.

In one embodiment the composition comprising a compound of formula (I) or (II), or a pharmaceutically acceptable derivative thereof for use as disclosed herein is an add-on therapy to existing therapies.

### Routes of Administration

It will be appreciated that the preferred route of administration will depend on the general condition and age of the subject to be treated, the nature of the condition to be treated, the location of the tissue to be treated in the body and the active ingredient chosen.

In one embodiment the composition comprising a compound of formula (I) or (II) as defined herein is administered by systemic administration, local administration, enteral administration or parenteral administration. Appropriate dosage forms for such administration may be prepared by conventional techniques.

### Systemic Administration

Systemic administration is capable of introducing the compound into the blood stream to ultimately target the sites of desired action.

Such routes of administration are any suitable routes, such as an enteral route, the oral, rectal, nasal, pulmonary, buccal, sublingual, transdermal, intracisternal, intraperitoneal, and parenteral (including subcutaneous, intramuscular, intrathecal, intravenous and intradermal) route.

In one embodiment, the composition comprising a compound of formula (I) or (II) as defined herein is administered by systemic administration.

### Oral Administration

Oral administration is a route of administration where a substance is taken through the mouth. Suspensions, syrups and solid oral dosage forms, such as tablets, capsules and the like, are commonly used.

In one embodiment, the composition comprising a compound of formula (I) or (II) as defined herein is administered by oral administration.

### Parenteral Administration

Parenteral administration is any administration route not being the oral route whereby the medicament avoids first-pass degradation in the liver. Accordingly, parenteral administration includes any injections and infusions, for example bolus injection or continuous infusion, such as intravenous administration, intramuscular administration and subcutaneous administration. Furthermore, parenteral administration includes inhalations and topical administration.

Accordingly, the compound may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the compound may be administered topically to cross the skin. The subcutaneous and intramuscular forms of parenteral administration are generally preferred.

### Local Treatment

The compound as disclosed herein is in one embodiment used as a local treatment, i.e. is introduced directly to the site(s) of action. Accordingly, the compound may be applied to the skin or mucosa directly, or the compound may be injected into the site of action, for example into the diseased tissue or to an end artery leading directly to the diseased tissue.

### Dosage

According to the present disclosure, the composition comprising a compound of formula (I) or (II) is administered to individuals in need of treatment in pharmaceutically effective doses. A therapeutically effective amount of a compound is an amount sufficient to cure, prevent, reduce the risk of, alleviate or partially arrest the clinical manifestations of a given disease and its complications. The amount that is effective for a particular therapeutic purpose will depend on the severity and the sort of the disorder as well as on the weight and general state of the subject. The compound may be administered one or several times per day, for example once a day, such as from 1 to 8 times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day. Alternatively, the compound may be administered less than once a day, such as once every second day, for example once every third day, such as once every fourth day, for example once every fifth day, such as once every sixth day, for example once every week.

All dosages of the pharmaceutically acceptable salts of AP1189 indicated herein are calculated as the free base, unless expressly indicated otherwise.

In one embodiment the composition comprising a compound of formula (I) as defined herein is administered in a therapeutically effective amount, such as in an amount of 1 mg to 1000 mg compound of formula (I) or (II) per day (calculated as the free base).

It follows that in one embodiment the compound is administered in an amount of 1 mg to 1000 mg, such as 1 to 5 mg, 5 to 10 mg, 10 to 15 mg, 15 to 20 mg, 20 mg, 20 to 30 mg, 30 to 40 mg, 40 to 50 mg, 50 to 60 mg, 60 to 70 mg, 70 to 80 mg, 80 to 90 mg, 90 to 100 mg, 100 to 110 mg, 110 to 120 mg, 120 to 130 mg, 130 to 140 mg, 140 mg to 150 mg, 150 mg to 160 mg, 160 to 170 mg, 170 to 180 mg, 180 to 190 mg, 190 to 200 mg, 200 to 240 mg, 240 to 280 mg, 280 to 320 mg, 320 to 360 mg, 360 to 400 mg, 400 to 440 mg, 440 to 500 mg, 500 to 560 mg, 560 to 620 mg, 620 to 680 mg, 680 to 740 mg, 740 to 800 mg, 800 to 860 mg, 860 to 920 mg, 920 to 980 mg, 980 to 1000 mg, for example 500 to 1000 mg per day (calculated as the free base).

In one embodiment the compound is (E)-N-trans-{3-[1-(2-nitrophenyl)-1H-pyrrol-2-yl]-allylidene}-aminoguanidinium acetate (AP1189; resomelagon) and is administered in an amount of 1 mg to 1000 mg, such as 1 to 5 mg, 5 to 10 mg, 10 to 15 mg, 15 to 20 mg, 20 mg, 20 to 30 mg, 30 to 40 mg, 40 to 50 mg, 50 to 60 mg, 60 to 70 mg, 70 to 80 mg, 80 to 90 mg, 90 to 100 mg, 100 to 110 mg, 110 to 120 mg, 120 to 130 mg, 130 to 140 mg, 140 mg to 150 mg, 150 mg to 160 mg, 160 to 170 mg, 170 to 180 mg, 180 to 190 mg, 190 to 200 mg, 200 to 240 mg, 240 to 280 mg, 280 to 320 mg, 320 to 360 mg, 360 to 400 mg, 400 to 440 mg, 440 to 500 mg, 500 to 560 mg, 560 to 620 mg, 620 to 680 mg, 680 to 740 mg, 740 to 800 mg, 800 to 860 mg, 860 to 920 mg, 920 to 980 mg, 980 to 1000 mg, for example 500 to 1000 mg per day (calculated as the acetate salt).

Per day means the dosage may be given in one dosage or divided in multiple dosages per day, including once a day (QD), twice a day (BID) and/or three times a day (TID).

In one embodiment the compound is administered in an amount of about 50 mg once daily, about 100 mg once daily, about 200 mg once daily, about 300 mg once daily, about 400 mg once daily, about 500 mg once daily, about 600 mg once daily, about 700 mg once daily, about 800 mg once daily, about 900 mg once daily or about 1000 mg once daily (calculated as the free base).

In one embodiment the compound is (E)-N-trans-{3-[1-(2-nitrophenyl)-1H-pyrrol-2-yl]-allylidene}-aminoguanidinium acetate (AP1189; resomelagon) and is administered in an amount of about 50 mg once daily, about 100 mg once daily, about 200 mg once daily, about 300 mg once daily, about 400 mg once daily, about 500 mg once daily, about 600 mg once daily, about 700 mg once daily, about 800 mg once daily, about 900 mg once daily or about 1000 mg once daily (calculated as the acetate salt).

In another embodiment the compound is administered in an amount of 0.01 mg/kg bodyweight to 40 mg/kg bodyweight, such as 0.01 mg/kg bodyweight to 0.05 mg/kg bodyweight, 0.05 to 0.1 mg/kg bodyweight, 0.1 to 0.5 mg/kg bodyweight, 0.5 mg to 1 mg/kg bodyweight, 1 to 2 mg/kg bodyweight, 2 to 3 mg/kg bodyweight, 3 to 5 mg/kg bodyweight, 5 to 10 mg/kg bodyweight, 10 to 15 mg/kg bodyweight, 15 to 20 mg/kg bodyweight, 20 to 30 mg/kg bodyweight, for example 30 to 40 mg/kg bodyweight.

### Formulation

In one embodiment the composition comprising a compound of formula (I) or (II) as defined herein is a pharmaceutical composition, such as a pharmaceutically safe composition.

The composition comprising a compound of formula (I) or (II)) as defined herein may be administered in any suitable way e.g. orally, sublingually, or parenterally, and it may be presented in any suitable form for such administration, e.g. in the form of solutions, suspension, aerosols, tablets, capsules, powders, syrups, implant or dispersions for injection.

In one embodiment, the composition comprising a compound of formula (I) or (II) as defined herein is formulated as a suspension.

In one embodiment, the composition comprising a compound of formula (I) or (II) as defined herein is formulated as an oral dosage form, such as a solid oral dosage form or pharmaceutical entity, such as tablets or capsules, or a liquid oral dosage form. Methods for the preparation of solid pharmaceutical preparations are well known in the art.

In one embodiment, the composition comprising a compound of formula (I) or (II) as defined herein comprises one or more excipients.

In another embodiment the composition comprising a compound of formula (I) or (II) as defined herein is formulated as an injectable dose form.

In one embodiment the composition comprising a compound of formula (I) or (II) as defined herein is formulated in the form of a solid pharmaceutical entity, suitably as a tablet or a capsule.

The compound (I) or (II) as the free base or the salt thereof may be administered alone or in combination with pharmaceutically acceptable carriers or excipients, in either single or multiple doses. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

### EXAMPLES

### Example 1

Acute peritonitis like other local inflammatory conditions such as acute respiratory distress syndrome (ARDS) develop into a life-threatening condition with development of systemic inflammatory distress syndrome (SIDS) and sepsis. Development of SIDS is associated with high circulating levels of pro-inflammatory cytokines including IL-1B and IL-6.

Acute peritonitis was induced by the injection of 1 mg zymosan A (Sigma-Aldrich) i.p. in 0.5 ml sterile PBS in mice. Twelve hours later the mice were sacrificed by CO₂ exposure, and peritoneal cavities were washed with 4 ml ice-cold PBS containing 3 mM EDTA. Cells were stained with Turk's solution (0.01% crystal violet in 3% acetic acid) and counted using a Neubauer hemocytometer or were stained with FITC-conjugated mAb for Ly-6G/Gr1, F4/80, and corresponding isotype controls (eBioscience, Hatfield, U.K.), and subjected to flow-cytometry analysis using a BD FACSCalibur platform (BD Biosciences, Oxford, U.K.).

Levels of IL-1B and IL-6 in quick frozen samples were quantified according to the manufacturer's instructions using ELISA Ready-SET-Go! (eBioscience).

All animal studies were approved by and performed under the guidelines of the Ethical Committee for the Use of Animals, Barts and The London School of Medicine and Home Office regulations (Guidance on the Operation of Animals, Scientific Procedures Act, 1986). Male (7-8 wk old) C57BL/6J wild-type (WT) mice were purchased from Charles River Laboratories.

Test Item A=AP1189 (E)-N-trans-{3-[1-(2-nitrophenyl)-1H-pyrrol-2-yl]-allylidene}-aminoguanidium acetate.

Test Item A, 1 mg/kg or vehicle treatment were given intraperitoneal 30 minutes before induction of the acute peritonitis. N=6 for in both groups.

### Results:

Pre-treatment with Test Item A reduced the levels of the pro-inflammatory cytokines IL-1β and IL-6 with 37% respectively 59% (p<0.05 vs vehicle) when compared to pretreatment with vehicle.

When compared to vehicle treatment neutrophil accumulation was reduced with 70% following pre-treatment with Test Item A.

### Example 2

A double-blind, multi-center, two-part, randomized, placebo-controlled study of the safety, tolerability, and efficacy of 4 weeks of treatment with AP1189 in early rheumatoid arthritis (RA) patients with active joint disease (Clinical phase IIa). The study population will consist of newly diagnosed subjects with severe active RA (CDAI (Clinical disease activity score)>22) who are to start up-titration with methotrexate (MTX).

Doses of 50 mg and 100 mg AP1189 are selected. The doses used in this Example are calculated based on the specific acetate salt form. Hence, a dosage of “100 mg AP1189” (acetate salt) as referred to herein correspond to a dosage of 83 mg AP1189 free base.

The peak respectively trough concentrations identified in the repeated dose part of the Phase I study with AP1189 were during steady conditions as follows:

| | C Max (mean +/− | C Trough (Mean +/− |
|---|---|---|
| | SD, N = 9) | SD, N = 9) |
| 50 mg once daily | 180 ± 38 ng/ml | 46 ± 14 ng/ml |
| 100 mg once daily | 427 ± 76 ng/ml | 100 ± 25 ng/ml |
| 200 mg once daily | 893 ± 198 ng/ml | 186 ± 45 ng/ml |

### Primary Safety Endpoint

The safety of AP1189 against placebo by evaluating adverse events (AEs), serious adverse events (SAEs), and laboratory abnormalities.

### Primary Efficacy Endpoint

The change in CDAI after 4 weeks of treatment compared to baseline will be evaluated by assessing the following, by treatment group:
Mean change in CDAI from baseline
Proportion of subjects with a change in CDAI score from severe (CDAI>22) to moderate (CDAI≤22) at week 4 compared to baseline.

### Secondary Efficacy Endpoints

The effects of AP1189 against placebo will be evaluated by assessing the following by treatment group:
Proportion of subjects achieving a reduction of more than 10 (ten) swollen and/or tender joints (SJC and TJC, summarized) at week 4 compared to baseline
Proportion of subjects achieving a change in CDAI score at week 4 compared to baseline
   Proportion of subjects with a 5-point decrease
   Proportion of subjects with a 10-point decrease
   Proportion of subjects with a 15-point decrease.
Proportion of subjects achieving a change in DAS28 from DAS28 >3.2 to DAS28 ≤3.2 at week 4 compared to baseline
Change of HAQ-DI at week 4 compared to baseline
Change of FACIT-Fatigue at week 4 compared to baseline.
Proportion of subjects achieving ACR response assessed by ACR 20, ACR 50, and AC70

### Tertiary Efficacy Endpoints

The effect of AP1189 compared to placebo at week 4 compared to baseline will be further evaluated by assessing the following by treatment group:
CXCL13, IL-1β, IL-6, IL-10, and TNF-α.
Synovial biopsy at baseline and after 4 weeks treatment (only Part 2 at selected sites).

### Study Design

This study is a multicenter, two-part, randomized, double-blind, placebo-controlled, 4-week study with repeated doses of AP1189. The study population will consist of newly diagnosed subjects with severe active RA (CDAI >22) who are to start up-titration with MTX. A minimum of 90 subjects are expected to complete the study; plus 45 subjects from Bulgaria and/or Moldova for a sub-study. Up to 120 subjects are planned to be enrolled to account for discontinuation rate, and up to 60 subjects for the sub-study.

Subjects who fulfill the enrollment criteria will be randomized in a 2:1 ratio in group A and B. One group will receive active treatment, and the other group will receive a placebo. Group C/D will have the same 2:1 ratio between active and placebo.
Group A (12 subjects): AP1189 dose 50 mg, once daily for 4 weeks (28 days) plus MTX (10-25 mg) weekly
Group B (6 subjects): placebo for 4 weeks (28 days) plus MTX (10-25 mg) weekly
Group C (12 subjects): AP1189 dose 100 mg, once daily for 4 weeks (28 days) plus MTX (10-25 mg) weekly
Group D (6 subjects): placebo for 4 weeks (28 days) plus MTX (10-25 mg) weekly

### Number of Subjects Part 1

A minimum of 24 subjects is expected to complete Part 1 of the study. About 32 subjects are planned to be enrolled in accounting for discontinuation rate.

### Part 2

All subjects will be randomized into one design only, either design 1, 2, or 3
Design 1: AP1189 dose 50 mg (min. 44 subjects) or placebo (min. 22 subjects), once daily for 4 weeks (28 days) plus MTX (10-25 mg) weekly
Design 2: AP1189 dose 100 mg (min. 44 subjects) or placebo (min. 22 subjects), once daily for 4 weeks (28 days) plus MTX (10-25 mg) weekly.
Design 3: Continue with the same doses as in Part 1, in a 1:1:1 ratio (AP1189 50 mg (min. 22 subjects), AP1189 100 mg (min. 22 subjects) or placebo (min. 22 subjects) plus MTX (10-25 mg) weekly

### Number of Subjects in Part 2

A minimum of 66 subjects is expected to complete Part 2 of the study, and 45 subjects in the sub-study. About 88 subjects are planned to be enrolled in the main study accounting for discontinuation rate, and about 60 subjects in the sub-study.

### Study Duration

Total study duration is 18 months, and the study duration for each subject is approximately and up to 10 weeks.

### Number of Investigational Sites

The study is to be conducted at sites in Europe, and Moldova.

### Study Population

The study population will consist of subjects with severe active RA, defined as CDAI >22, who are about to begin up-titration with MTX.

The study drug information is presented in the below tables.

TABLE 1
Test Treatment

| | |
|---|---|
| Name | AP1189 |
| Dosage strength | 50 mg, 100 mg |
| Formulation | Powder (for suspension) |
| Route of | Oral |
| Supplier | SynAct Pharma |

TABLE 2
Reference Treatment

| | |
|---|---|
| Name | Placebo |
| Dosage strength | 0 mg |
| Formulation | Powder |
| Route of | Oral |
| Supplier | SynAct Pharma |

### Description of AP1189 Product:

IUPAC: E-N-[trans-3-{1-(2-nitrophenyl)-1H-pyrrole-2-yl}-allylideneamino]guanidinium acetate. Priorietary: AP1189. WHO generic name: Resomelagon.

The substance is an acetic acid salt that appears as an odorless, yellow to brownish solid. The molecular weight is 358.35 for the acetate salt and 298.30 for the free base.

### Methotrexate (MTX)

All subjects will follow the local guideline for starting treatment with MTX and continue MTX treatment throughout the study.

### Laboratory Assessments

The latest updated reference ranges from the local laboratory will be used to identify subjects with clinically notable laboratory values.

### Hematology

Hemoglobin, white blood cell (WBC) count (total and differential: leukocytes, neutrophils, eosinophils, basophils, lymphocytes, monocytes), red blood cells (RBC), thrombocytes and hemoglobin A1c (HbA1C). The hematology blood samples will be taken at screening, baseline, after 2 weeks and 4 weeks treatment.

### Biochemistry

Sodium, potassium, chloride, calcium, glucose, creatinine, urea, albumin, unconjugated and total bilirubin, aspartate transaminase (AST), alanine transaminase (ALT), alkaline phosphatase (ALP), gamma-glutamyl transferase (GGT) and INR. The biochemistry blood samples will be taken at all visits.

### Thyroid Function

Thyroxine (T4) free, triiodothyronine (T3) total or free, and the thyroid-stimulating hormone (TSH). Blood samples for measuring the thyroid function.

### Urinalysis

A dipstick urine test for blood, protein, and glucose will be performed at the site at the Screening Visit. A urine sample may be sent for urine culture.

### Serology

RF or anti-CCP, HBsAg, HBV antibody and HCV antibody. RF is an antibody that is detectable in the blood of approximately 80% of adults with RA.

CRP is an acute phase reactant, a protein made by the liver and released into the blood within a few hours after tissue injury, the start of an infection, or other cause of inflammation. The CRP will most often be increased by inflammation. One of the aims of treatment is to reduce the CRP to normal levels. CRP will be measured at screening, baseline, after 2 weeks and 4 weeks treatment.

### Safety (AE and SAE)

Safety measures (AEs, SAEs, including laboratory abnormalities and vital signs such as blood pressure) will be registered during the whole study duration.

### Safety Assessments (Sub-Study, Only Part 2)

The arthroscopy sub-study in Part 2 (only selected sites), will assess the effect of 4 weeks treatment with AP1189/placebo compared to baseline by examining synovial fluid: (evaluating the change in the percentage of polymorphs, monocytes, and lymphocytes in synovial fluid).

### Efficacy Assessments

### Swollen Joint Count (SJC) and Tender Joint Count (TJC)

An assessment of 66 joints for swelling and 68 joints for tenderness will be made at screening, baseline, after 2 weeks and 4 weeks treatment. Joints will be assessed and classified as swollen/not swollen and tender/not tender by pressure and joint manipulation on physical examination.

### Clinical Disease Activity Index (CDAI)

The CDAI is a clinical composite score for following patients with RA.

Descriptive changes in CDAI where CDAI=SJC (28)+TJC (28)+PGA+IGA;
SJC (28): Swollen 28-Joint Count (shoulders, elbows, wrists, MCPs, PIPs including thumb IP, knees);
TJC (28): Tender 28-Joint Count (shoulders, elbows, wrists, MCPs, PIPs including thumb IP, knees);
PGA: Patient Global Disease Activity (patient's self-assessment of overall RA disease activity on a scale 0-100 where 100 is maximal activity);
IGA: Physician's Global Disease Activity (evaluator's assessment of the subject's overall RA disease activity on a scale 0-100 where 100 is maximal activity)

The CDAI will be scored at screening, baseline, after 2 weeks and 4 weeks treatment, and at the final visit (one week after final dose).

### Disease Activity Score 28 (DAS28)

The DAS28 is a combined index for measuring disease activity in RA. The index includes swollen and tender joint counts, CRP, and general health status. In this trial CRP will be used to calculate the DAS28 score. The index is calculated using the following formula:

$$DAS 28 - CRP ( 4 ) = 0.56 * sqrt ( TJC 28 ) + 0.28 * sqrt ( SJC 28 ) + 0.36 * ln ( CRP + 1 ) + 0.014 * GH + 0.96$$

Where, TJC=tender joint count on 28 joints, SJC=swollen joint count on 28 joints, ln=natural log, CRP=C-reactive Protein, and GH=general health, i.e., patient's global assessment of disease activity (100-mm VAS).

The DAS28 will be scored at baseline, after 2 weeks and 4 weeks treatment, and at the final visit.

### Physician's Global Assessment of Disease Activity VAS (“Investigator Global VAS”)

The physician's assessment of the subject's current disease activity on a 100 mm horizontal VAS. Investigator Global VAS will be measured at screening, baseline, after 2 weeks and 4 weeks treatment.

### Patient's Global Assessment of Disease Activity VAS (“Patient Global VAS”)

The subject's overall assessment of their current disease activity on a 100 mm horizontal VAS. Patient Global VAS will be measures at screening, baseline, after 2 weeks and 4 weeks treatment.

### Patient's Assessment of Pain VAS (“Patient Pain VAS”)

The subject's assessment of his/her current level of pain on a 100 mm horizontal VAS. Patient Pain VAS will be measured at screening, baseline, after 2 weeks and 4 weeks treatment.

### Quality of Life and Physical Function

### Functional Assessment of Chronic Illness Therapy-Fatigue (FACIT-Fatigue)

The FACIT-Fatigue assessment is a 13-item questionnaire with subjects scoring each item on a 5-point scale. FACIT-Fatigue will be scored at baseline, after 2 weeks and 4 weeks treatment.

### Health Assessment Questionnaire-Disability Index (HAQ-DI)

HAQ-DI is a validated tool to evaluate physical function. HAQ-DI will be scored at baseline, after 2 weeks and 4 weeks treatment.

### American College of Rheumatology Response Rates

The ACR (American College of Rheumatology) Criteria is a standard criterion to measure the effectiveness of various arthritis medications or treatments in clinical trials for RA.

The ACR response rates ACR20, ACR50, and ACR70 are defined as ≥20%, ≥50% and ≥70% improvement, respectively, in swollen and tender joint counts (SJC/TJC) and 3 of the following 5 assessments: Patient's Global Assessment of Disease Activity (see above), Physician's Global Assessment of Disease Activity (see above), Patient's Assessment of Pain (see above), Health Assessment Questionnaire (HAQ-DI, see above), and C-Reactive Protein (CRP).

### Pharmacokinetic Assessments

### PK Sample Collection

Plasma PK samples for exposure-response analysis will be taken after 1, 2, 3- and 4-weeks treatment.

### Cytokine Samples

Plasma samples for CXCL13, IL-1B, IL-6, IL-10, and TNF-α analysis will be taken at baseline, after 2 weeks and 4 weeks treatment.

### Biobanking

The following samples are kept at frozen storage in a biobank or similar, as applicable per country, during the study:
Cytokines (CXCL13, IL-13, IL-6, IL-10, and TNF-α), plasma
PK, plasma

Analysis of all cytokines and PK will be performed at a central laboratory. The samples will be sent and analyzed on a regular basis.

### Example 3

Results from the clinical trial described in Example 2 are presented here. The available data show a significant effect of AP1189 in reducing CDAI for both tested dosages, i.e. 50 mg and 100 mg. Results are indicated at 2 weeks (mid-treatment period), at 4 weeks (end of treatment period), and at 5 weeks (one week follow-up after last dosage).

| | | Base- | | | | | Difference |
|---|---|---|---|---|---|---|---|
| CDAI | Screening | line | Week 2 | Week 4 | Week 5 | Difference | in % |
| 50 mg | 35.6 | 36.1 | 29.9 | 24.2 | 22.9 | −11.9 | 33% |
| AP1189 | | | | | | | |
| (n = 31) | | | | | | | |
| Group | | | | | | | |
| mean | | | | | | | |
| Standard | 12 | 11.1 | 10.4 | 10.4 | 11.3 | — | — |
| deviation | | | | | | | |
| 100 mg | 38.9 | 39 | 28.9 | 23.9 | 21.3 | −15.1 | 39% |
| AP1189 | | | | | | | |
| (n = 31) | | | | | | | |
| Group | | | | | | | |
| mean | | | | | | | |
| Standard | 9.3 | 9 | 10.9 | 13 | 11.9 | — | — |
| deviation | | | | | | | |
| Placebo | 35.3 | 36.3 | 28.3 | 27.4 | 23.2 | −8.9 | 25% |
| (n = 32) | | | | | | | |
| Group | | | | | | | |
| mean | | | | | | | |
| Standard | 8.4 | 8.6 | 12.3 | 13.6 | 13.8 | — | — |
| deviation | | | | | | | |

The effect of treatment on blood pressure is shown in the table below. For placebo an increase is observed in MAP, SAP, and DAP of 1.2 mmHg, 1.0 mmHg, and 1.3 mmHg respectively after 4 weeks treatment. On the contrary MAP decreased by 0.8 mmHg for the patients treated with 50 mg AP1189 and 1.6 mmHg for the 100 mg group. Similarly, SAP decreased by 3.1 mmHg and DAP by 0.8 mmHg for the 100 mg AP1189 group, and 2.2 mmHg and 0.1 respectively for the 50 mg group. The number of observed abnormalities in blood pressure was reduced in both treatment groups, whereas no change was observed in the placebo group.

| | | | | SAP | DAP | MAP |
|---|---|---|---|---|---|---|
| Blood | | | | Average/ | Average/ | Average/ |
| Pressure | Base Line | n | CNA | Median | Median | Median |
| Placebo | | 31 | 1 (3%) | 125.6 | 79.5 | 94.9 |
| 50 mg AP1189 | | 33 | 4 (11%) | 131.3 | 80.0 | 97.1 |
| 100 mg AP1189 | | 34 | 4 (11%) | 129.9 | 81.1 | 97.4 |
| Blood | | | | | | |
| Pressure | 4 Weeks | n | | | | |

| | | | | | |
|---|---|---|---|---|---|
| Placebo | 31 | 1 (3%) | 126.6 | 80.8 | 96.1 |
| 50 mg AP1189 | 33 | 2 (6%) | 129.1 | 79.9 | 96.3 |
| 100 mg AP1189 | 34 | 1 (3%) | 126.8 | 80.3 | 95.8 |

CNA = clinically notable vital sign abnormalities (SAP ≥160, <90; DAP ≥105, <50), SAP = systolic arterial pressure (mmHg), DAP = diastolic arterial pressure (mmHg), MAP = mean arterial pressure (DAP + ⅓(SAP − DAP)) (mmHg).

The baseline characteristics of the study participants are comparable between the groups:

| | Placebo | | AP1189 50 mg | | AP1189 100 mg | |
|---|---|---|---|---|---|---|
| | (n = 34) | | (n = 35) | | (n = 35) | |
| Age | 56.4 ± 13.1 | | 56.4 ± 13.1 | | 55.3 ± 13.9 | |
| | 61 | (range: 26-78) | 57 | (range: 28-79) | 56.5 | (range: 27-77) |
| Age 18-64: | 73.5% | | 71.4% | | 72.2% | |
| Age 65-84: | 26.5% | | 28.5% | | 27.8% | |
| Weight | 75.2 ± 19.7 | | 75.9 ± 17.7 | | 79.3 ± 16.7 | |
| | 71.6 | (range: 48-145) | 75.3 | (range: 42-111) | 76.6 | (range: 48-118) |

| Height | 167.5 ± 7.8 | 167.5 ± 6.6 | | 116.3 ± 8.4 | |
|---|---|---|---|---|---|
| | 167 (range: 155-185) | 167 | (range: 157-183) | 165.5 | (range: 151-185) |

| | | | |
|---|---|---|---|
| Ethnicity | 34 White | 34 White, 1 Asian | 35 White, 1 Other |
| Gender | 79.4% Females | 77.1% Females | 77.8% Females |

| | | AP1189 | AP1189 |
|---|---|---|---|
| | Placebo | 50 mg | 100 mg |
| | (n = 32) | (n = 31)* | (n = 35)* |
| Clinical Disease | 36.3 ± 8.6 | 36.1 ± 11.0 | 39 ± 9 |
| Activity Index (CDAI): | | | |
| QVAS-Patient: | 6.2 ± 1.2 | 6.3 ± 1.3 | 6.4 ± 1.3 |
| QVAS-Investigator: | 6.6 ± 1.8 | 5.9 ± 2.3 | 5.8 ± 2.1 |
| Swollen Joints (century) | 10.0 ± 3.9 | 10.3 ± 4.4 | 11.8 ± 5.0 |
| Tender Joints (century) | 13.9 ± 5.6 | 13.7 ± 6.4 | 15.0 ± 5.8 |
| DAS28 Score | 5.5 ± 1.0 | 5.4 ± 0.9 | 5.7 ± 0.9 |
| CRP (mg/L) | 19.1 ± 26.5 | 14.9 ± 18.4 | 26.1 ± 40.7 |

1. A composition comprising a compound of formula (I): 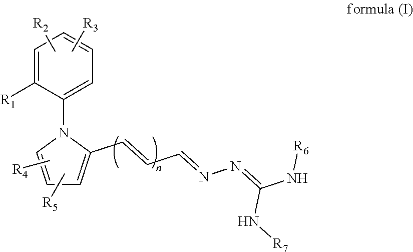
including tautomeric and stereoisomeric forms thereof; wherein n is 1; and
R₁ is CF₃, CCl₃, F, Cl, NO₂ or CN, and R₂, R₃, R₄, R₅, R₆, and R₇ are hydrogen;
or a pharmaceutically acceptable derivative thereof,
for use in the treatment of cardiovascular disease, hypertension and/or atherosclerosis.

2. The composition for use according to claim 1, wherein said compound is for use in the treatment, in the prevention, in reducing the risk of developing, and/or in ameliorating said cardiovascular disease, hypertension and/or atherosclerosis.

3. The composition for use according to any of the preceding claims, wherein the compound is of formula (II): 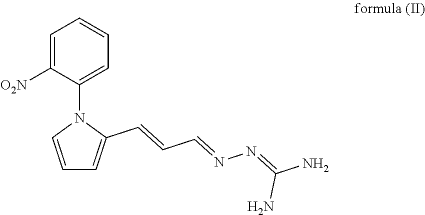 [SMILES: NC(N)=N/N=C/C=C/c1cccn1-c1ccccc1[N+](=O)[O-]]
including tautomeric and stereoisomeric forms thereof; or a pharmaceutically acceptable derivative thereof.

4. The composition for use according to any of the preceding claims, for use in the treatment of cardiovascular disease.

5. The composition for use according to any of the preceding claims, wherein said cardiovascular disease is selected from the group consisting of coronary artery diseases (CAD) such as angina and myocardial infarction; stroke, heart failure, hypertensive heart disease, atherosclerotic heart disease, rheumatic heart disease, cardiac hypertrophy, cardiomyopathy, abnormal heart rhythms, atrial fibrillation, congenital heart disease, congestive heart failure, valvular heart disease, carditis, aortic aneurysms, peripheral artery disease, vascular disease, thromboembolic disease, and venous thrombosis.

6. The composition for use according to any of the preceding claims, for use in the treatment of atherosclerosis.

7. The composition for use according to any of the preceding claims, for use in a method of reducing atheromatous plaques and/or reducing fatty streaks.

8. The composition for use according to any of the preceding claims, wherein said composition reduces atheromatous plaques, reduces fatty streaks and/or reduces thrombus formation.

9. The composition for use according to any of the preceding claims, for use in a method of reducing the risk of one or more of thrombus formation,
thromboembolism, and thrombus formation or thromboembolism resulting in ischemia and/or infarction in individuals with atherosclerosis.

10. The composition for use according to any of the preceding claims, for use in the treatment of atherosclerotic cardiovascular disease (ASCVD).

11. The composition for use according to any of the preceding claims, for use in the treatment of an atherosclerotic cardiovascular disease selected from the group consisting of coronary artery disease, stroke (cerebrovascular disease), and peripheral artery disease.

12. The composition for use according to any of the preceding claims, for use in the treatment of vascular inflammation.

13. The composition for use according to any of the preceding claims, wherein said composition reduces vascular inflammation.

14. The composition for use according to any of the preceding claims, for use in the treatment of hypertension.

15. The composition for use according to any of the preceding claims, for use in the treatment of hypertensive heart diseases, such as selected from the group consisting of atherosclerotic cardiovascular disease including coronary artery disease, cerebrovascular disease and peripheral artery disease; cardiac hypertrophy, coronary artery disease, congestive heart failure, atrial fibrillation and aortic aneurism.

16. The composition for use according to any of the preceding claims, for use in a method of reducing blood pressure.

17. The composition for use according to any of the preceding claims, wherein said composition reduces blood pressure.

18. The composition for use according to any of the preceding claims, wherein said composition reduces mean arterial blood pressure (MAP).

19. The composition for use according to any of the preceding claims, wherein said composition reduces mean arterial blood pressure (MAP) at least about 0.5 mmHg, such as at least about 0.6 mmHg, such as at least about 0.7 mmHg, such as at least about 0.8 mmHg, such as at least about 0.9 mmHg, such as at least about 1.0 mmHg, such as at least about 1.2 mmHg, such as at least about 1.4 mmHg, such as at least about 1.6 mmHg, such as at least about 1.8 mmHg, such as at least about 2.0 mmHg.

20. The composition for use according to any of the preceding claims, wherein said composition reduces mean arterial blood pressure (MAP) about 0.5 to about 0.8 mmHg, such as about 0.8 to about 1.0 mmHg, such as about 1.0 to about 1.2 mmHg, such as about 1.2 to about 1.4 mmHg, such as about 1.4 to about 1.6 mmHg, such as about 1.6 to about 1.8 mmHg, such as about 1.8 to about 2.0 mmHg.

21. The composition for use according to any of the preceding claims, wherein said composition reduces systolic arterial blood pressure (SAP).

22. The composition for use according to any of the preceding claims, wherein said composition reduces systolic arterial blood pressure (SAP) at least about 1.5 mmHg, such as at least about 2.0 mmHg, such as at least about 2.1 mmHg, such as at least about 2.2 mmHg, such as at least about 2.3 mmHg, such as at least about 2.4 mmHg, such as at least about 2.5 mmHg, such as at least about 3.0 mmHg, such as at least about 3.1 mmHg, such as at least about 3.2 mmHg, such as at least about 3.3 mmHg, such as at least about 3.4 mmHg, such as at least about 3.5 mmHg

23. The composition for use according to any of the preceding claims, wherein said composition reduces systolic arterial blood pressure (SAP) about 1.5 to about 2.0 mmHg, such as about 2.0 to about 2.1 mmHg, such as about 2.1 to about 2.2 mmHg, such as about 2.2 to about 2.3 mmHg, such as about 2.3 to about 2.4 mmHg, such as about 2.4 to about 2.5 mmHg, such as about 2.5 to about 3.0 mmHg, such as about 3.0 to about 3.1 mmHg, such as about 3.1 to about 3.2 mmHg, such as about 3.2 to about 3.3 mmHg, such as about 3.3 to about 3.4 mmHg, such as about 3.4 to about 3.5 mmHg

24. The composition for use according to any of the preceding claims, wherein said composition reduces diastolic arterial blood pressure (DAP).

25. The composition for use according to any of the preceding claims, wherein said composition reduces diastolic arterial blood pressure (DAP) at least about 0.1 mmHg, such as at least about 0.2 mmHg, such as at least about 0.3 mmHg, such as at least about 0.4 mmHg, such as at least about 0.5 mmHg, such as at least about 0.6 mmHg, such as at least about 0.7 mmHg, such as at least about 0.8 mmHg, such as at least about 0.9 mmHg, such as at least about 1.0 mmHg, such as at least about 1.2 mmHg, such as at least about 1.4 mmHg, such as at least about 1.6 mmHg, such as at least about 1.8 mmHg, such as at least about 2.0 mmHg.

26. The composition for use according to any of the preceding claims, wherein said composition reduces diastolic arterial blood pressure (DAP) about 0.1 to about 0.2 mmHg, such as about 0.2 to about 0.3 mmHg, such as about 0.3 to about 0.4 mmHg, such as about 0.4 to about 0.5 mmHg, such as about 0.5 to about 0.6 mmHg, such as about 0.6 to about 0.7 mmHg, such as about 0.7 to about 0.8 mmHg, such as about 0.9 to about 1.0 mmHg.

27. The composition for use according to any of the preceding claims, wherein said composition clinically notable vital sign abnormalities (CNA) (systolic arterial pressure ≥160, <90 mmHg; diastolic arterial pressure ≥105, <50 mmHg).

28. The composition for use according to any of the preceding claims, for use in an individual with high blood pressure.

29. The composition for use according to any of the preceding claims, for use in an individual with hypertension, such as stage 1 hypertension or stage 2 hypertension.

30. The composition for use according to any of the preceding claims, for use in an individual with atherosclerosis and/or atheromatous plaques.

31. The composition for use according to any of the preceding claims, for use in an individual with a cardiovascular disease, or an individual with an increased risk of developing a cardiovascular disease.

32. The composition for use according to any of the preceding claims, for use in an individual with arthritic disease.

33. The composition for use according to any of the preceding claims, for use in an individual with rheumatoid arthritis.

34. The composition for use according to any of the preceding claims, for use in an individual with rheumatoid arthritis and cardiovascular comorbidities.

35. The composition for use according to any of the preceding claims, wherein said compound is selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof.

36. The composition for use according to any of the preceding claims, wherein said pharmaceutically acceptable derivative thereof is a pharmaceutically acceptable salt of an inorganic acid or an organic acid.

37. The composition for use according to claim 36, wherein said organic acid is selected from the group consisting of: formic acid, acetic acid, trichloroacetic acid, trifluoroacetic acid, propionic acid, benzoic acid, cinnamic acid, citric acid, fumaric acid, glycolic acid, lactic acid, maleic acid, malic acid, malonic acid, mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid and p-toluenesulfonic acid.

38. The composition for use according to any claims 36-37, wherein said organic acid is acetic acid, succinic acid, tartaric acid or propionic acid.

39. The composition for use according to claim 38, wherein said organic acid is succinic acid.

40. The composition for use according to claim 38, wherein said organic acid is acetic acid.

41. The composition for use according to claim 36, wherein said inorganic acid is selected from the group consisting of: hydrochloric acid, hydrobromic acid, hydroiodic acid, phosphoric acid, sulphuric acid and nitric acid.

42. The composition for use according to any of the preceding claims, wherein said compound is selected from the group consisting of {3-[1-(2-nitrophenyl)-1H-pyrrol-2-yl]-allylidene}-aminoguanidinium succinate and (E)-N-trans-{3-[1-(2-nitrophenyl)-1H-pyrrol-2-yl]-allylidene}-aminoguanidinium succinate.

43. The composition for use according to any of the preceding claims, wherein said compound is (E)-N-trans-{3-[1-(2-nitrophenyl)-1H-pyrrol-2-yl]-allylidene}-aminoguanidinium succinate.

44. The composition for use according to any of the preceding claims, wherein said compound is selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate.

45. The composition for use according to any of the preceding claims, wherein said compound is (E)-N-trans-{3-[1-(2-nitrophenyl)-1H-pyrrol-2-yl]-allylidene}-aminoguanidinium acetate (resomelagon; AP1189).

46. The composition for use according to any of the preceding claims, wherein said compound is administered in an amount of 1 mg to 1000 mg per day, such as 1 to 5 mg, 5 to 10 mg, 10 to 15 mg, 15 to 20 mg, 20 to 30 mg, 30 to 40 mg, 40 to 50 mg, 50 to 60 mg, 60 to 70 mg, 70 to 80 mg, 80 to 90 mg, 90 to 100 mg, 100 to 110 mg, 110 to 120 mg, 120 to 130 mg, 130 to 140 mg, 140 mg to 150 mg, 150 mg to 160 mg, 160 to 170 mg, 170 to 180 mg, 180 to 190 mg, 190 to 200 mg, 200 to 240 mg, 240 to 280 mg, 280 to 320 mg, 320 to 360 mg, 360 to 400 mg, 400 to 440 mg, 440 to 500 mg, 500 to 560 mg, 560 to 620 mg, 620 to 680 mg, 680 to 740 mg, 740 to 800 mg, 800 to 860 mg, 860 to 920 mg, 920 to 980 mg, 980 to 1000 mg, for example 500 to 1000 mg per day.

47. The composition for use according to any of the preceding claims, wherein said compound is administered in an amount of about 50 mg once daily, about 100 mg once daily, about 200 mg once daily, about 300 mg once daily, about 400 mg once daily, about 500 mg once daily, about 600 mg once daily, about 700 mg once daily, about 800 mg once daily, about 900 mg once daily or about 1000 mg once daily.

48. The composition for use according to any of the preceding claims, wherein said composition is pharmaceutically safe.
